(19) United States
(12) Patent Application Publication
(10) Pub. No.: US 2026/0337167 A1
(43) Pub. Date: Oct. 29, 2026

(54) ELIMINATION REACTION FOR PREPARING SUBSTITUTED ALKENES

(71) Applicant: Bayer Aktiengesellschaft, Leverkusen (DE)

(72) Inventors: Anton Lishchynskyi, Solingen (DE); Sergii Pazenok, Leichlingen (DE); Mark James Ford, Oberreifenberg (DE); Frank Memmel, Guntersblum (DE); Andreas Rembiak, Langerwehe (DE); Wahed Ahmed Moradi, Monheim (DE)

(21) Appl. No.: 19/104,436

(22) PCT Filed: Aug. 15, 2023
(86) PCT No.: PCT/EP2023/072429

§ 371 Date: Feb. 18, 2025

(30) Foreign Application Priority Data
Aug. 19, 2022 (EP) 22191153.0

(51) Int. Cl. C07D 261/04

(52) U.S. Cl. CPC C07D 261/04

(57) ABSTRACT

The present invention relates to a novel process for preparing substituted alkenes of the formula (A), especially isoxazoline-□□□-vinylcarboxylic acid derivatives of the formula (I).

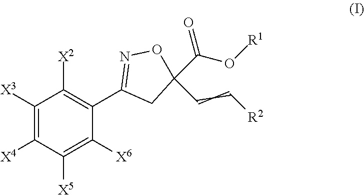

The present invention relates to a process for preparing substituted alkenes of the formula (A), especially a process for preparing isoxazoline-5,5-vinylcarboxylic acid derivatives of the formula (I) and (V).

The preparation of substituted alkenes is an essential process step in a wide variety of synthetic processes in the agrochemical and pharmaceutical industry. Substituted alkenes can usually only be prepared at high cost using harsh conditions.

In particular, isoxazoline-5,5-vinylcarboxylic acid derivatives of the general formula (I) are important precursors of active agrochemical ingredients (cf. WO2018/228985). WO2018/228985 already describes a process for preparing isoxazoline-5,5-vinylcarboxylic acid derivatives of the general formula (I). However, the process described therein is only suitable for an industrial scale synthesis to a limited extent due to the use of reactants that are difficult to obtain on an industrial scale, for example trifluoromethanesulfonic anhydride or diazabicycloundecene (DBU).

It was thus an object of the invention to provide an elimination reaction for preparing substituted alkenes of the formula (A), in particular for preparing isoxazoline-5,5-vinylcarboxylic acid derivatives of the formula (I), which is suitable for synthesis on an industrial scale, and nevertheless has a high yield and selectivity, such that laborious purification methods can be dispensed with.

In a first aspect, this object according to the invention is achieved by a process for preparing substituted alkenes of the formula (A)

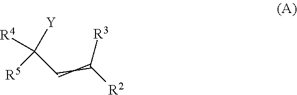

in which
R² is H or alkyl,
R³ is H or alkyl,
Y is CO₂R¹, CN, CHO, CF₃,
where R¹ is C₁-C₈-alkyl, cycloalkyl, unsubstituted benzyl, unsubstituted phenyl or mono- or di-C₁-C₃-alkyl-substituted benzyl or phenyl;
R⁴, R⁵ are each independently alkyl, cycloalkyl, aryl, heteroaryl, in each case unsubstituted or substituted, or a heteroatom;
   or R⁴ and R⁵, together with the carbon atom linking R⁴ and R⁵ in the compounds of the formula (A), form a cycloalkyl, aryl, or heterocyclyl, in each case unsubstituted or substituted, preferably a substituted isoxazoline, particularly preferably a phenyl-substituted isoxazoline,
characterized in that compounds of the general formula (B)

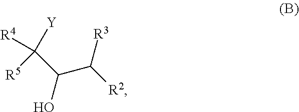

in which R², R³, R⁴, R⁵ and Y have the definitions given above,
are reacted with a compound of the formula R^{F}SO₂F in the presence of a base and a Lewis acid,
where R^{F} is selected from F, CF₃, C₂F₅, C₃F₇, C₄F₉, CF₂Cl, C₆F₅, alkoxy-CF₂, R⁶OCOCF₂SO₂F where R⁶ is defined as C₁-C₈-alkyl or cycloalkyl (step 1).

By the process according to the invention, the compounds of the formula (A) are obtained in high selectivity and yield. There is likewise no need to use any reagents that are difficult to obtain on an industrial scale. Surprisingly, it has been found that the use of R^{F}SO₂F together with a Lewis acid could reduce the formation of undesirable fluorinated secondary components. Due to the chemical selectivity of the process according to the invention, no laborious purification of the product of the formula (A) is required and downstream reactions can be carried out without complex intermediate purification or directly in the reaction mixture. This is particularly important when the undesirable secondary components are difficult or impossible to separate from the desired compounds.

R^{F}SO₂F is preferably SO₂F₂.

The gas SO₂F₂ may be prepared cost-effectively and is therefore well-suited for industrial scale synthesis in accordance with the present invention.

Suitable bases according to the invention are preferably selected from trimethylamine, triethylamine, tripropylamine, tributylamine, N,N-diisopropylethylamine, N,N-dimethylcyclohexylamine, 2-methyl-5-ethylpyridine, pyridine, 3,5-dimethylpyridine, 2,4,6-trimethylpyridine, 2-methylpyridine, 3-methylpyridine, N,N-dimethylacetamide, N,N-dimethylformamide, N,N-dibutylformamide, N-methylimidazole (NMI), N-butylimidazole (NBI), 1,3-dimethyl-2-imidazolidinone (DMEU), tetramethylurea (TMU), more preferably from triethylamine, tripropylamine, tributylamine, N,N-diisopropylethylamine or N,N-dimethylcyclohexylamine.

As base, particular preference is given to triethylamine, N,N-dimethylcyclohexylamine, or N,N-diisopropylethylamine. In particular, the use of these bases results in high conversions of the compounds of the formula (B).

N,N-Dimethylcyclohexylamine is especially preferred.

The base is preferably used in amounts of at least 1.8 equivalents, more preferably 2.0 equivalents, particularly preferably of at least 2.5 equivalents, based on one equivalent of the compounds of the formula (B).

Preferably, the base may also be used as solvent for the process according to the invention.

Alternatively, if another solvent is used in addition to the base, the base may be used in amounts of at most 10.0 equivalents, more preferably at most 5.0 equivalents, based on one equivalent of the compounds of the formula (B).

The base is preferably used in amounts between 1.8 and 10, preferably 2.0 and 5.0 equivalents, based on one equivalent of the compounds of the formula (B).

Suitable Lewis acids according to the invention are preferably selected from BF₃, PF₅, and SbF₅.

In particular, BF₃ is a suitable Lewis acid, which may be present as a free gas or as a solution, preferably in acetonitrile, in aliphatic or cyclic ether compounds, as an etherate complex or as a complex with an amine base, more preferably with triethylamine.

Preferably, BF₃ is introduced as a gas, which allows simple process control, or alternatively preferably reacted as a solution in acetonitrile.

It is particularly preferable to select SO₂F₂ as R^{F}SO₂F and BF₃ as the Lewis acid. This combination has proven to be cost-effective and allows high conversion with significant suppression of the formation of undesirable fluorinated secondary components.

The Lewis acid is preferably used in amounts of at least 0.8 equivalents, more preferably at least 1.0 equivalent, based on one equivalent of the compounds of the formula (B).

The Lewis acid is further preferably used in amounts of at most 3.0 equivalents, more preferably at most 1.5 equivalents, based on one equivalent of the compounds of the formula (B).

The Lewis acid is further preferably used in amounts of 0.8 equivalents to 3.0 equivalents, more preferably 1.0 to 1.5 equivalents, based on one equivalent of the compounds of the formula (B).

The compounds of the formula (B) are reacted according to the invention with R^{F}SO₂F.

R^{F}SO₂F is preferably used here in amounts of at least 0.8 equivalents, more preferably at least 1.0 equivalent, particularly preferably at least 1.2 equivalents, based on one equivalent of the compounds of the formula (B).

R^{F}SO₂F is further preferably used in amounts of at most 4.0 equivalents, more preferably at most 3.0 equivalents, particularly preferably at most 1.5 equivalents, based on one equivalent of the compounds of the formula (B).

R^{F}SO₂F is further preferably used in amounts of 0.8 equivalents to 4.0 equivalents, more preferably in amounts of 1.0 to 3.0 equivalents, even more preferably in amounts of 1.2 to 1.5 equivalents, based on one equivalent of the compounds of the formula (B).

Alternatively, R^{F}SO₂F may also be used in significant excess. Unreacted R^{F}SO₂F may be reacted again in further processes. This applies particularly to SO₂F₂ which is introduced as a gas.

Step 1 is preferably carried out at a reaction temperature in the range of −20 °C to 120 °C, more preferably in the range of 20 °C to 100 °C.

More preferably, step 1 is carried out at least partially at a temperature in the range between 60 °C to 100 °C. The elevated temperature can further increase the conversion of the reaction.

In particular, step 1 is preferably carried out at an initial temperature of 10 °C to 30 °C and at a terminal temperature of 60 °C to 100 °C.

The reaction time for step 1 is preferably in the range from 6 h to 40 h, particularly preferably in the range from 10 to 30 h.

More preferably, step 1 is carried out at a temperature in the range between 60 °C to 100 °C for a period of at least 5 hours, more preferably at least 10 hours, particularly preferably at least 15 h.

More preferably, step 1 is carried out at a temperature in the range between 60 °C to 100 °C for a period of 10 to 30 h.

The reaction of step 1 is preferably carried out in the region of standard pressure (1013 hPa).

Optionally, the reaction may alternatively be conducted under elevated or reduced pressure.

For example, the reaction may preferably be carried out in the range of 300 hPa to 5000 hPa or 500 hPa to 2000 hPa, preferably in the range of 1013 hPa±200 hPa.

The reaction of step 1 is preferably carried out in a solvent. Suitable solvents here are particularly acetonitrile, propionitrile, butyronitrile, acetone, N,N-dimethylacetamide, N-methylpyrrolidinone (NMP), N,N-dimethylformamide (DMF), dimethylpropyleneurea (DMPU), toluene, xylene, tetrahydrofuran (THF), isopropyl acetate (iPrOAc), dimethyl carbonate, diethyl carbonate, ethylene carbonate, propylene carbonate (4-methyl-1,3-dioxolan-2-one), ethyl acetate (EtOAc), methyl tert-butyl ether (MTBE), cyclopropyl methyl ether (CPME), methyl-THF, 4-methyltetrahydropyran (methyl-THP), tert-amyl methyl ether (TAME), dihydrolevoglucosenone (cyrene), N,N-dimethylcyclohexylamine or mixtures thereof in any ratios.

Other nitrogen-containing solvents or organic bases may be used in addition to the aforementioned bases N,N-dimethylacetamide, N,N-dimethylformamide or N,N-dimethylcyclohexylamine.

Particular preference is given to acetonitrile, toluene, xylene, tetrahydrofuran (THF), isopropyl acetate (i-PrOAc), or N,N-dimethylcyclohexylamine.

Step 1 may alternatively be carried out in an excess of the (liquid) base without the use of an additional solvent. In this case, step 1 is carried out particularly in an excess of one of the aforementioned bases.

In a second aspect, the object according to the invention is achieved by a process for preparing isoxazoline-5,5-vinylcarboxylic acid derivatives of the formula (I),

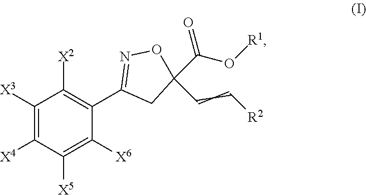

in which
X² is H, C₁-C₄-alkyl, C₁-C₄-fluoroalkyl, C₁-C₄-fluoroalkoxy, C₁-C₄-alkoxy, fluorine or CN,
X³ is H, C₁-C₄-alkyl, C₁-C₄-fluoroalkyl, C₁-C₄-fluoroalkoxy, C₁-C₄-alkoxy, fluorine, chlorine or CN,
X⁴ is H, C₁-C₄-alkyl, C₁-C₄-fluoroalkyl, C₁-C₄-fluoroalkoxy, C₁-C₄-alkoxy, fluorine or CN,
X⁵ is H, C₁-C₄ alkyl, C₁-C₄ fluoroalkyl, C₁-C₄ fluoroalkoxy, C₁-C₄ alkoxy, fluorine, chlorine or CN,
X⁶ is H, C₁-C₄-alkyl, C₁-C₄-fluoroalkyl, C₁-C₄-fluoroalkoxy, C₁-C₄-alkoxy, fluorine or CN,
R¹ is C₁-C₈-alkyl, cycloalkyl, unsubstituted benzyl, unsubstituted phenyl or mono- or di-C₁-C₃-alkyl-substituted benzyl or phenyl;
R² is H or alkyl,
characterized in that compounds of the general formula (IV)

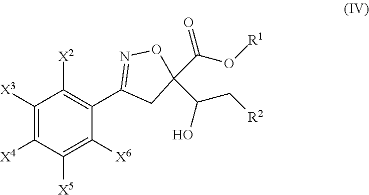

in which R¹, R², X² to X⁶ have the definitions given above,
are reacted with a compound of the formula R^{F}SO₂F in the presence of a base and a Lewis acid, where R^{F} is selected from F, CF₃, C₂F₅, C₃F₇, C₄F₉, CF₂Cl, C₆F₅, alkoxy-CF₂, R⁶OCOCF₂SO₂F where R⁶ is defined as C₁-C₈-alkyl or cycloalkyl (step 1).

The compounds of the formula (I) are also obtained in high yields; an industrial-scale synthesis can be designed cost-effectively.

The method features described above and their technical effects on the conversion of the compounds of the formula (B) to (A) apply equally to the conversion of the compounds (IV) to (I):

R^{F}SO₂F is preferably SO₂F₂.

Suitable bases according to the invention are preferably selected from trimethylamine, triethylamine, tripropylamine, tributylamine, N,N-diisopropylethylamine, N,N-dimethylcyclohexylamine, 2-methyl-5-ethylpyridine, pyridine, 3,5-dimethylpyridine, 2,4,6-trimethylpyridine, 2-methylpyridine, 3-methylpyridine, N,N-dimethylacetamide, N,N-dimethylformamide, N,N-dibutylformamide, N-methylimidazole (NMI), N-butylimidazole (NBI), 1,3-dimethyl-2-imidazolidinone (DMEU), tetramethylurea (TMU), more preferably from triethylamine, tripropylamine, tributylamine, N,N-diisopropylethylamine or N,N-dimethylcyclohexylamine.

As base, particular preference is given to triethylamine, N,N-dimethylcyclohexylamine, or N,N-diisopropylethylamine.

The base is preferably used in amounts of at least 1.8 equivalents, more preferably of at least 2.0 equivalents, particularly preferably of at least 2.5 equivalents, based on one equivalent of the compounds of the formula (IV).

Preferably, the base may also be used as solvent for the process according to the invention. If another solvent is used in addition to the base, the base may be used in amounts of at most 10.0 equivalents, more preferably at most 5.0 equivalents, based on one equivalent of the compounds of the formula (IV). The base may be used particularly in amounts between 1.8 and 10, preferably 2.0 and 5.0 equivalents, based on one equivalent of the compound of the formula (IV).

Suitable Lewis acids according to the invention are preferably selected from BF₃, PF₅, and SbF₅.

In particular, BF₃ is a suitable Lewis acid, which may be present as a free gas or as a solution, preferably in acetonitrile, in aliphatic or cyclic ether compounds, as an etherate complex or as a complex with an amine base, more preferably with triethylamine.

Preferably, BF₃ is introduced as a gas, which allows simple process control, or alternatively preferably reacted as a solution in acetonitrile.

It is particularly preferable to select SO₂F₂ as R^{F}SO₂F and BF₃ as the Lewis acid.

The Lewis acid is preferably used in amounts of at least 0.8 equivalents, more preferably at least 1.0 equivalent, based on one equivalent of the compounds of the formula (IV).

The Lewis acid is further preferably used in amounts of at most 3.0 equivalents, more preferably at most 1.5 equivalents, based on one equivalent of the compounds of the formula (IV).

The Lewis acid is further preferably used in amounts of 0.8 equivalents to 3.0 equivalents, more preferably 1.0 to 1.5 equivalents, based on one equivalent of the compounds of the formula (IV).

The compounds of the formula (IV) are reacted according to the invention with R^{F}SO₂F.

R^{F}SO₂F is preferably used here in amounts of at least 0.8 equivalents, more preferably at least 1.0 equivalent, particularly preferably at least 1.2 equivalents, based on one equivalent of the compounds of the formula (IV).

R^{F}SO₂F is further preferably used in amounts of at most 4.0 equivalents, more preferably at most 3.0 equivalents, particularly preferably at most 1.5 equivalents, based on one equivalent of the compounds of the formula (IV).

R^{F}SO₂F is further preferably used in amounts of 0.8 equivalents to 4.0 equivalents, more preferably in amounts of 1.0 to 3.0 equivalents, even more preferably in amounts of 1.2 to 1.5 equivalents, based on one equivalent of the compounds of the formula (IV).

Alternatively, R^{F}SO₂F may also be used in significant excess. Unreacted R^{F}SO₂F may be reacted again in further processes. This applies particularly to SO₂F₂ which is introduced as a gas.

Step 1 is preferably carried out at a reaction temperature in the range of −20 °C to 120 °C, more preferably in the range of 20 °C to 100 °C.

More preferably, step 1 is carried out at least partially at a temperature in the range between 60 °C to 100 °C. The elevated temperature can further increase the conversion of the reaction.

In particular, step 1 is preferably carried out at an initial temperature of 10 °C to 30 °C and at a terminal temperature of 60 °C to 100 °C.

The reaction time for step 1 is preferably in the range from 6 to 40 h, particularly preferably in the range from 10 to 30 h.

More preferably, step 1 is carried out at a temperature in the range between 60 °C to 100 °C for a period of at least 5 hours, more preferably at least 10 hours, particularly preferably at least 15 h.

More preferably, step 1 is carried out at a temperature in the range between 60 °C to 100 °C for a period of 10 to 30 h.

The reaction of step 1 is preferably carried out in the region of standard pressure (1013 hPa).

Optionally, the reaction may alternatively be conducted under elevated or reduced pressure.

For example, the reaction may preferably be carried out in the range of 300 hPa to 5000 hPa or 500 hPa to 2000 hPa, preferably in the range of 1013 hPa±200 hPa.

The reaction of step 1 is preferably carried out in a solvent. Suitable solvents here are particularly acetonitrile, propionitrile, butyronitrile, acetone, N,N-dimethylacetamide, N-methylpyrrolidinone (NMP), N,N-dimethylformamide (DMF), dimethylpropyleneurea (DMPU), toluene, xylene, tetrahydrofuran (THF), isopropyl acetate (iPrOAc), dimethyl carbonate, diethyl carbonate, ethylene carbonate, propylene carbonate (4-methyl-1,3-dioxolan-2-one), ethyl acetate (EtOAc), methyl tert-butyl ether (MTBE), cyclopropyl methyl ether (CPME), methyl-THF, 4-methyltetrahydropyran (methyl-THP), tert-amyl methyl ether (TAME), dihydrolevoglucosenone (cyrene), N,N-dimethylcyclohexylamine or mixtures thereof in any ratios.

Other nitrogen-containing solvents or organic bases may be used in addition to the aforementioned bases N,N-dimethylacetamide, N,N-dimethylformamide or N,N-dimethylcyclohexylamine.

Particular preference is given to acetonitrile, toluene, xylene, tetrahydrofuran (THF), isopropyl acetate (i-PrOAc), or N,N-dimethylcyclohexylamine.

Step 1 may alternatively be carried out in an excess of the (liquid) base without the use of an additional solvent. In this case, step 1 is carried out particularly in an excess of one of the aforementioned bases.

The preferred embodiments described below refer, if appropriate, to all formulae described herein.

Preferred radical definitions for X² to X⁶ are as follows:
X² is H, methyl, trifluoromethyl, difluoromethyl, difluoromethoxy, trifluoromethoxy, fluorine, methoxy or CN,
X³ is H, methyl, trifluoromethyl, difluoromethyl, difluoromethoxy, trifluoromethoxy, fluorine, chlorine, methoxy or CN,
X⁴ is H, methyl, trifluoromethyl, difluoromethyl, difluoromethoxy, trifluoromethoxy, fluorine, methoxy or CN,
X⁵ is H, methyl, trifluoromethyl, difluoromethyl, difluoromethoxy, trifluoromethoxy, fluorine, chlorine, methoxy or CN,
X⁶ is H, methyl, trifluoromethyl, difluoromethyl, difluoromethoxy, trifluoromethoxy, fluorine, methoxy or CN.

Particularly preferred radical definitions for X² to X⁶ are as follows:
X² is H,
X³ is H, methyl, trifluoromethyl, difluoromethyl, fluorine, chlorine, methoxy or CN,
X⁴ is fluorine, H,
X⁵ is H, methyl, trifluoromethyl, difluoromethyl, fluorine, chlorine, methoxy or CN,
X⁶ is H.

Very particularly preferred radical definitions for X² to X⁶ are as follows:
X² is H,
X³ is H or fluorine,
X⁴ is H or fluorine,
X⁵ is H or fluorine,
X⁶ is H.

Most preferred radical definitions for X² to X⁶ are as follows:
X² is H,
X³ is fluorine,
X⁴ is H,
X⁵ is fluorine,
X⁶ is H.

With regard to the further configurations of the invention:
R¹ is preferably methyl, ethyl, isopropyl, n-propyl, 2-methyl-1-propyl, 1-methyl-1-propyl, cyclohexyl, 3-methyl-1-butyl, 1-butyl, 1-pentyl, benzyl or tert-butyl, particularly preferably isopropyl, 2-methyl-1-propyl, 1-methyl-1-propyl, cyclohexyl, 3-methyl-1-butyl, 1-butyl or 1-pentyl, especially preferably isopropyl, 2-methyl-1-propyl, 1-methyl-1-propyl, cyclohexyl, 3-methyl-1-butyl, and most preferably isopropyl or 2-methyl-1-propyl.
R² is preferably H, methyl or ethyl.

Other preferred radical definitions are as follows:
R¹ is methyl, ethyl, isopropyl, n-propyl, 2-methyl-1-propyl, 1-methyl-1-propyl, cyclohexyl, 3-methyl-1-butyl, 1-butyl, 1-pentyl, benzyl or tert-butyl,
R² is H, methyl or ethyl,

Other particularly preferred radical definitions are as follows:
R¹ is methyl, ethyl, isopropyl, n-propyl, 2-methyl-1-propyl, 1-methyl-1-propyl, cyclohexyl, 3-methyl-1-butyl, 1-butyl or 1-pentyl,
R² is H, methyl or ethyl,

Other very particularly preferred radical definitions are as follows:
R¹ is methyl, ethyl, isopropyl, n-propyl, 2-methyl-1-propyl, 1-methyl-1-propyl, 3-methyl-1-butyl or 1-butyl,
R² is H or methyl,

Other most preferred radical definitions are as follows:
R¹ is methyl, ethyl, isopropyl, n-propyl, 2-methyl-1-propyl or 1-butyl,
R² is H,

Other preferred radical definitions for the compounds of the formulae (I), (III), (IV) and (IV) are as follows:
X² is H, methyl, trifluoromethyl, difluoromethyl, difluoromethoxy, trifluoromethoxy, fluorine, methoxy or CN,
X³ is H, methyl, trifluoromethyl, difluoromethyl, difluoromethoxy, trifluoromethoxy, fluorine, chlorine, methoxy or CN,
X⁴ is H, methyl, trifluoromethyl, difluoromethyl, difluoromethoxy, trifluoromethoxy, fluorine, methoxy or CN,
X⁵ is H, methyl, trifluoromethyl, difluoromethyl, difluoromethoxy, trifluoromethoxy, fluorine, chlorine, methoxy or CN,
X⁶ is H, methyl, trifluoromethyl, difluoromethyl, difluoromethoxy, trifluoromethoxy, fluorine, methoxy or CN,
R¹ is methyl, ethyl, isopropyl, n-propyl, 2-methyl-1-propyl, 1-methyl-1-propyl, cyclohexyl, 3-methyl-1-butyl, 1-butyl, 1-pentyl, benzyl or tert-butyl,
R² is H, methyl or ethyl,

Other particularly preferred radical definitions are as follows:
X² is H,
X³ is H, methyl, trifluoromethyl, difluoromethyl, fluorine, chlorine, methoxy or CN,
X⁴ is fluorine, H,
X⁵ is H, methyl, trifluoromethyl, difluoromethyl, fluorine, chlorine, methoxy or CN,
X⁶ is H,
R¹ is methyl, ethyl, isopropyl, n-propyl, 2-methyl-1-propyl, 1-methyl-1-propyl, cyclohexyl, 3-methyl-1-butyl, 1-butyl or 1-pentyl,
R² is H, methyl or ethyl,

Other very particularly preferred radical definitions are as follows:
X² is H,
X³ is H or fluorine,
X⁴ is H or fluorine,
X⁵ is H or fluorine,
X⁶ is H,
R¹ is methyl, ethyl, isopropyl, n-propyl, 2-methyl-1-propyl, 1-methyl-1-propyl, 3-methyl-1-butyl or 1-butyl,
R² is H or methyl,

Other most preferred radical definitions are as follows:
X² is H,
X³ is fluorine,
X⁴ is H,
X⁵ is fluorine,
X⁶ is H,
R¹ is methyl, ethyl, isopropyl, n-propyl, 2-methyl-1-propyl or 1-butyl,
R² is H,

In a further particular configuration of the invention, the process according to the invention according to the second aspect further comprises the preparation of the compounds of the formula (IV) by reacting compounds of the formula (III)

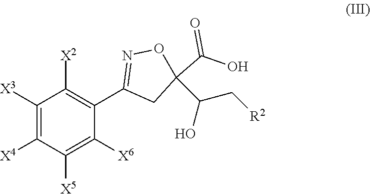

in which
R² and X² to X⁶ have the definitions given above,
with compounds of the formula R¹—OH in which R¹ has the definition given above. (Step 0-1)

In a further particular configuration of the invention, the compounds of the formula (I)

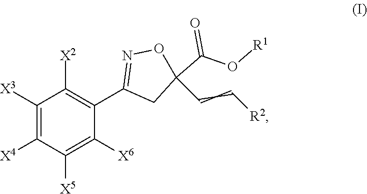

in which R¹, R² and X² to X⁶ have the definitions given above
are also hydrolysed in the presence of a base and then protonated in the presence of an acid or alternatively hydrolysed in the presence of an acid to give compounds of the formula (V)

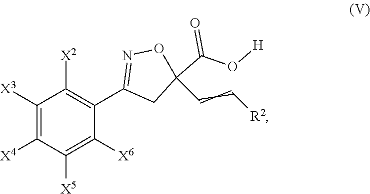

in which R² and X² to X⁶ have the meanings given above. (Step 2)

The compounds of the formulae (I), (III), (IV) and (V) may take the form of isomeric mixtures: The isomeric ratio between (Ia) and (Ib), (IIIa) and (IIIb), (IVa) and (IVb) and (Va) and (Vb) varies here. The same applies to the compounds of the formulae (A) and (B).

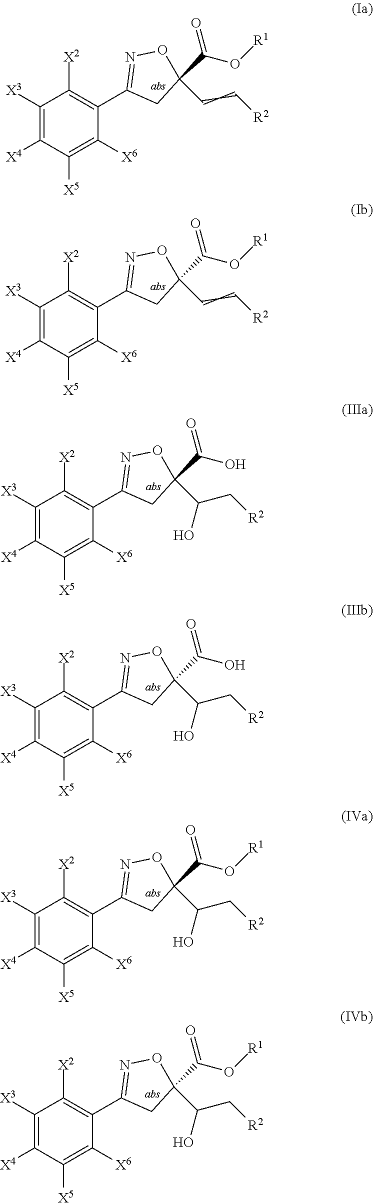

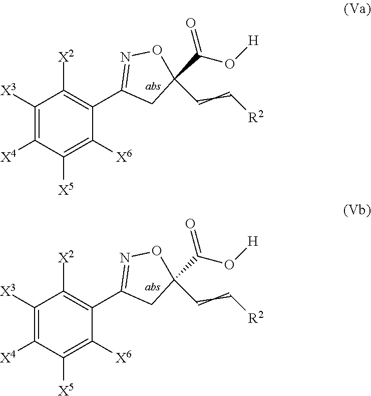

The terms used here are known to the person skilled in the art. Otherwise, the following definitions are used:

The C—C double bond [symbol] represents a cis or trans configuration of the respective radicals. This means that, for example, compounds of the formula (A)

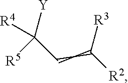

are understood to mean the configurations

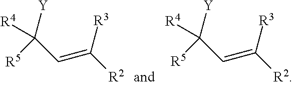

The statement “ . . . equivalents based on one equivalent” refers to the ratio of the molar amounts of the respective compounds. If, for example, the base is used in an amount of 1.8 equivalents based on one equivalent of a compound of the formula (B), this corresponds to a ratio of 1.8 mol 15 to 1 mol.

In the context of the present invention, unless defined differently elsewhere, the term “alkyl”, according to the invention, either on its own or else in combination with further terms, for example haloalkyl, is understood to mean a radical of a saturated, aliphatic hydrocarbon group which may be branched (isoalkyl, containing at least one secondary or tertiary or quaternary carbon atom in the alkyl chain) or unbranched (n-alkyl). “Alkyl” is understood to mean both unsubstituted and substituted alkyl radicals.

The term “alkoxy”, either on its own or else in combination with further terms, for example haloalkoxy, is understood in the present case to mean an O-alkyl radical, where the term “alkyl” is as defined above.

According to the invention, unless defined differently elsewhere, the term “cycloalkyl”, either on its own or else in combination with further terms, is understood to mean a C₃-C₈-cycloalkyl radical, for example cyclopropyl, cyclobutyl, cyclopentyl, cyclohexyl, cycloheptyl and cyclooctyl. “Cycloalkyl” is understood to mean both unsubstituted and substituted cycloalkyl radicals.

Halogen-substituted radicals, for example fluoroalkyl, are mono- or polyhalogenated, up to the maximum number of possible substituents.

According to the invention, unless defined differently elsewhere, the term “aryl”, either on its own or else in combination with further terms, is understood to mean a monocyclic or polycyclic, preferably a monocyclic or bicyclic, aromatic hydrocarbon radical having preferably 6, 10 or 14 carbon atoms. An aryl radical may be unsubstituted or monosubstituted or polysubstituted, by identical or different substituents. Examples of suitable aryl radicals are phenyl, 1-naphthyl, 2-naphthyl and anthracenyl.

The term “heteroaryl” signifies a monocyclic or polycyclic, preferably a mono-, bi- or tricyclic, aromatic hydrocarbon radical having preferably 5, 6, 7, 8, 9, 10, 11, 12, 13 or 14 carbon atoms, particularly preferably having 5, 6, 9, 10, 13 or 14 carbon atoms, especially preferably having 5 or 6 carbon atoms, in which one or more carbon atoms have been replaced in each case by a heteroatom independently selected from the group consisting of oxygen, sulfur and nitrogen (NH). Heteroaryl radicals may have, as ring member(s), preferably 1, 2, 3, 4 or 5, particularly preferably 1, 2 or 3, heteroatom(s) each independently selected from the group consisting of oxygen, sulfur and nitrogen (NH). A heteroaryl radical may be unsubstituted or monosubstituted or polysubstituted, by identical or different substituents. Examples of suitable heteroaryl radicals include indolizinyl, benzimidazolyl, tetrazolyl, triazinyl, isoxazolyl, phthalazinyl, carbazolyl, carbolinyl, diazanaphthyl, thienyl, furyl, pyrrolyl, pyrazolyl, pyrazinyl, pyranyl, triazolyl, pyridinyl, imidazolyl, indolyl, isoindolyl, benzo[b]furanyl, benzo[b]thiophenyl, benzo[d]thiazolyl, benzodiazolyl, benzotriazolyl, benzoxazolyl, benzisoxazolyl, thiazolyl, thiadiazolyl, oxazolyl, oxadiazolyl, pyridazinyl, pyrimidinyl, indazolyl, quinoxalinyl, quinazolinyl, quinolinyl, naphthridinyl and isoquinolinyl.

In the context of the present invention, aryl or heteroaryl radicals may be fused with a mono- or bicyclic ring system. Examples of aryl radicals that are fused with a mono- or bicyclic ring system include (2,3)-dihydrobenzo[b]thiophenyl, (2,3)-dihydro-1H-indenyl, indolinyl, (2,3)-dihydrobenzofuranyl, (2,3)-dihydrobenzo[d]oxazolyl, benzo[d][1,3]dioxolyl, benzo[d][1,3]oxathiolyl, isoindolinyl, (1,3)-dihydroisobenzofuranyl, (1,3)-dihydrobenzo[c]thiophenyl, (1,2,3,4)-tetrahydronaphthyl, (1,2,3,4)-tetrahydroquinolinyl, chromanyl, thiochromanyl, (1,2,3,4)-tetrahydroisoquinolinyl, (1,2,3,4)-tetrahydroquinoxalinyl, (3,4)-dihydro-2H-benzo[b][1,4]oxazinyl, (3,4)-dihydro-2H-benzo[b][1,4]thiazinyl, (2,3)-dihydrobenzo[b][1,4]dioxinyl, (2,3)-dihydrobenzo[b][1,4]oxathiinyl, (6,7,8,9)-tetrahydro-5H-benzo[7]annulenyl, (2,3,4,5)-tetrahydro-1H-benzo[b]azepinyl and (2,3,4,5)-tetrahydro-1H-benzo[c]azepinyl.

The term “heterocyclyl” generally refers to cyclic systems having at least two different elements, particularly N or O, or N and O in combination with ring-forming carbon atoms. The heterocyclic systems may be saturated or aromatic, and monosubstituted or polysubstituted. An example is isoxazoline, preferably substituted.

If one of the aforementioned radicals is mono- or polysubstituted, suitable substituents are all those familiar to the person skilled in the art, preferably those which are each independently selected from the group consisting of F, Cl, Br, I, —NO₂, —CN, —OH, —SH, —NH₂, C₁-C₄-alkyl, C₁-C₄-fluoroalkyl, C₁-C₄-fluoroalkoxy, C₁-C₄-alkoxy, fluoroalkoxy, —N(C₁₋₅-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), —NH—C(═O)—O—C₁₋₅-alkyl, —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁-s-alkyl, —S(═O)₂-phenyl, —S(═O)₂—NH₂ and —SO₃H.

The ranges specified above generally or in preferred ranges apply correspondingly to the overall process. These definitions can be combined with one another as desired, i.e. including combinations between the respective preferred ranges.

Preference is given in accordance with the invention to using processes in which there is a combination of the meanings and ranges specified above as being preferred.

Particular preference is given in accordance with the invention to using processes in which there is a combination of the meanings and ranges specified above as being particularly preferred.

Very particular preference is given in accordance with the invention to using processes in which there is a combination of the meanings and ranges specified above as being very particularly preferred.

The greatest preference is given in accordance with the invention to using processes in which there is a combination of the meanings and ranges specified above as being most preferred.

### Elucidation of the Processes and Intermediates

### Step 0-1

The process according to the invention may comprise a step 0-1 in which the compounds of the formula (IV)

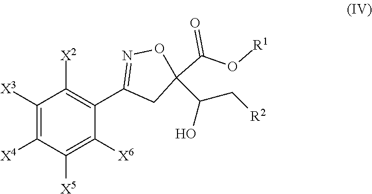

in which R¹, R² and X² to X⁶ have the definitions given above
are prepared by reacting compounds of formula (III)

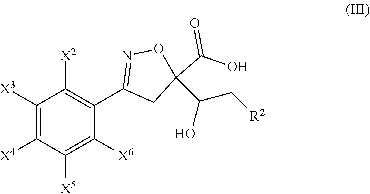

in which R², and X² to X⁶ have the definitions given above,
with compounds of the formula R¹—OH in which R¹ has the definition given above.

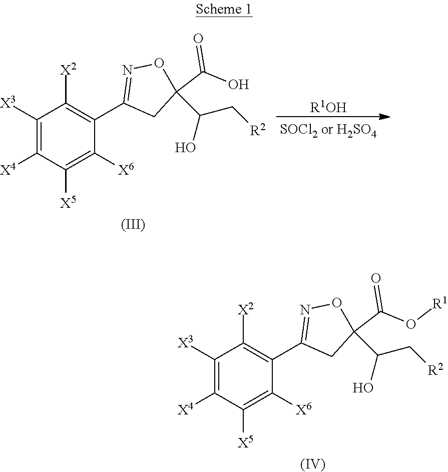

The preparation of compounds of the formula (III) is described, for example, in WO 2018/228985.

The esterification of the compound (III) with alcohols of the formula R¹—OH to give compound (IV) can be effected, for example, in the presence of 1.0 to 1.3 equivalents of thionyl chloride or catalytic amounts of sulfuric acid, based on one equivalent of the compounds of the formula (III), at 0 to 80 °C (at standard pressure) for 1.5 to 3 h. Preference is given here to using the compounds of the formula R¹—OH as reactant and solvent in a distinct excess of, for example, 4 to 8 equivalents.

The esterification of the compound (III) with alcohols of the formula R¹—OH to give compound (IV) can generally be carried out under any conditions known for such reactions in the prior art.

The compounds of the formula (IV) can be isolated by suitable workup steps generally known to those skilled in the art and characterized further, and can subsequently be used in step 1.

### Step 1

The preferred process according to the invention comprises a step 1, in which the compounds of the formula (I)

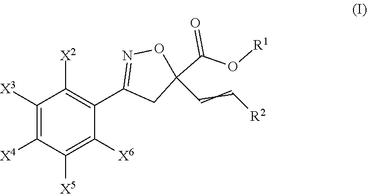

in which R¹, R², and X² to X⁶ have the definitions given above,
are prepared by reacting compounds of the formula (IV)

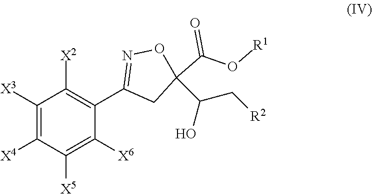

in which R¹, R² and X² to X⁶ have definitions given above
with R^{F}SO₂F, where R^{F} has the definition given above, in the presence of a base and a Lewis acid such as BF₃.

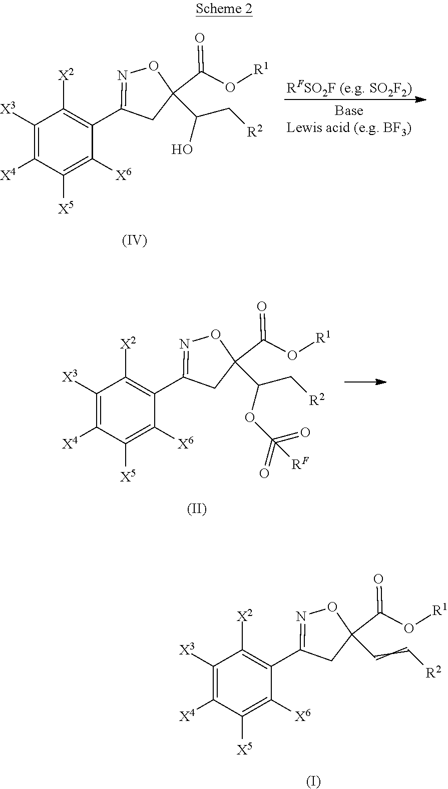

In this step, compounds of the formula (II) form as intermediates, which are increasingly converted to compounds of the formula (I) during the reaction.

### Step 2

The process according to the invention may further comprise the hydrolysis of compounds of the formula (I) to give compounds of the formula (V)

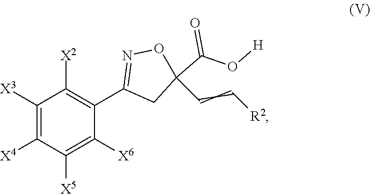

in which R² and X² to X⁶ have the definitions given above, in the presence of a base with subsequent protonation in the presence of an acid, or alternatively hydrolysis in the presence of an acid.

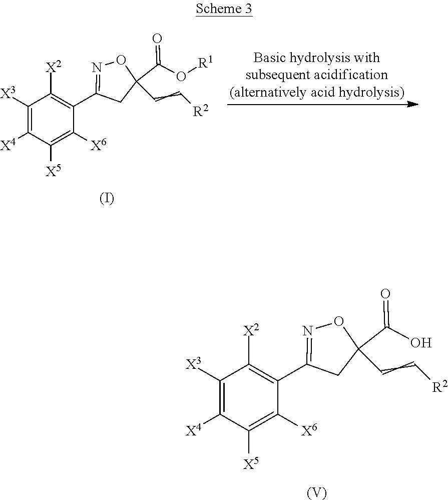

Suitable bases are especially inorganic bases, for example carbonates (e.g. (NH₄)₂CO₃, Li₂CO₃, Na₂CO₃, K₂CO₃, CaCO₃, MgCO₃), hydrogencarbonates (e.g. NH₄HCO₃, LiHCO₃, NaHCO₃, KHCO₃) or hydroxides (e.g. LiOH, NaOH, KOH, Ca(OH)₂); particular preference is given here to alkali metal or alkaline earth metal hydroxides, especially preferably KOH or NaOH.

The base is preferably used in the form of an aqueous solution at concentrations of 1-50% by weight, more preferably of an aqueous solution at concentrations of 5-45% by weight, most preferably of an aqueous solution at concentrations of 5-35% by weight.

The reaction with the base is preferably conducted at a reaction temperature in the range of 0 °C to 90 °C, particularly preferably in the range of 10 °C to 80 °C and especially preferably in the range of 15 °C to 60 °C.

The reaction is preferably conducted in the region of standard pressure (1013 hPa), for example in the range from 300 hPa to 5000 hPa or from 500 hPa to 2000 hPa, preferably as in the range of 1013 hPa±200 hPa.

The hydrolysis reaction time is preferably in the range of 0.5 h to 10 h.

The hydrolysis of the compounds of the formula (I) to give compounds (V) can generally be conducted under any conditions known for such reactions in the prior art.

The compounds of the formula (I) can be isolated by suitable workup steps generally known to those skilled in the art, for example by extraction and optionally distillation, and characterized further.

In general, step 1 may be followed, rather than by the hydrolysis (step 2), also by a transesterification of the compound of the formula (I) at the R¹ position.

Alternatively, step 2 may also be performed in the presence of an acid.

### Overall Process

In an advantageous configuration, the process according to the invention comprises steps 0-1 and 1, particularly advantageously 0-1, 1 and 2.

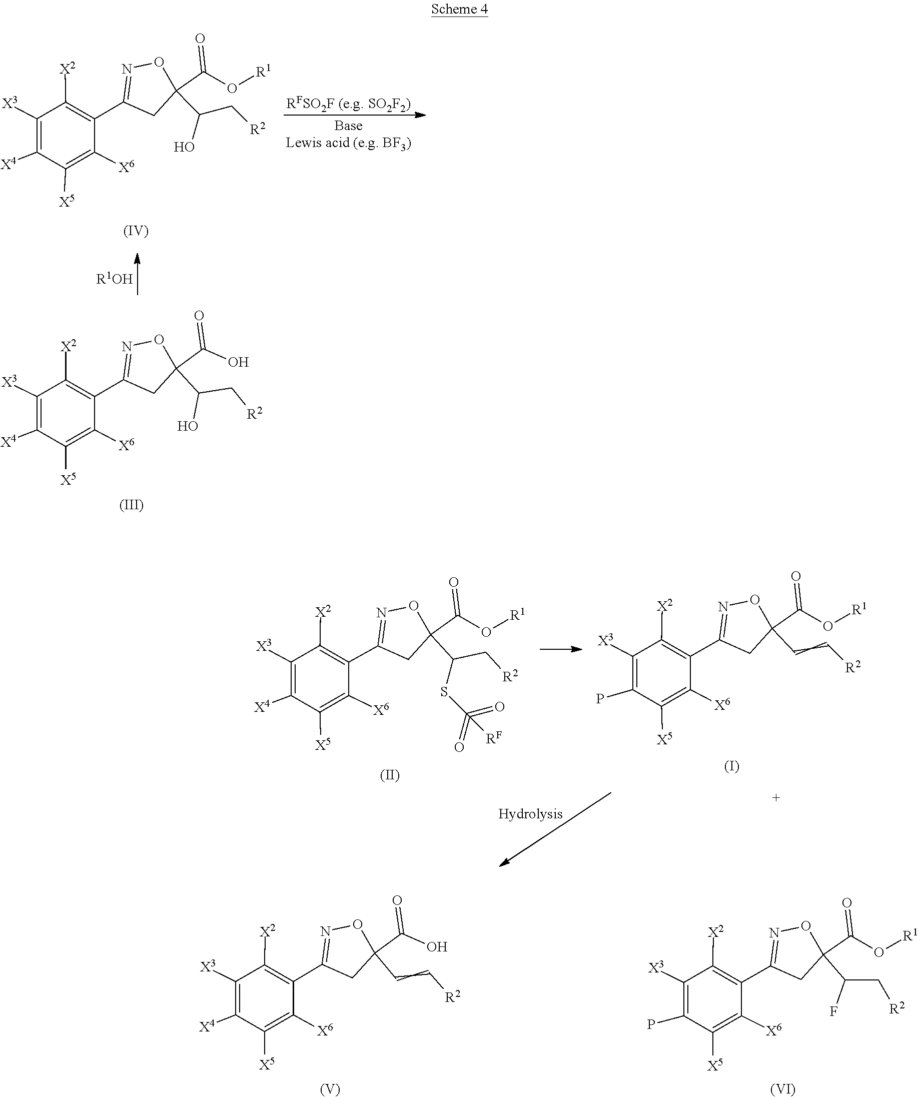

Scheme 4 gives a schematic overall representation of the process according to the invention, with all optional and obligatory steps. Reaction conditions and reactants are selected here in accordance with the above-described inventive and preferred configurations. All variables in the formulae are defined as described above.

The compounds of the formula (IV) and (I) may be isolated and optionally also purified before being used in the respective next synthetic step. However, it is also possible that the compounds are used directly in the next step without isolation and purification. In this case, the solvent and excess reagents from the preceding stage are removed by standard methods before the compounds are used in the next synthetic step.

### EXAMPLES

The present invention is elucidated in more detail by the examples which follow, without restricting the invention thereto.

### Measurement Methods

The products were characterized by ¹H-NMR and ¹⁹F-NMR spectroscopy and HPLC (high-performance liquid chromatography).

The NMR spectra were determined using a Bruker Avance 400 fitted with a flow probe head (volume 60 μl). The NMR data of the examples are listed in conventional form (δ values, multiplet splitting, number of hydrogen or fluorine atoms).

The solvent and the frequency at which the NMR spectrum was recorded are stated in each case. HPLC (High Performance Liquid Chromatography) was carried out on an Agilent 1100 LC system with the following parameters:

### a) for Reaction Monitoring (HPLC Area %)

Column: 150×4.6 mm, stainless steel; stationary phase: Phenomenex, Luna 5 μm C18 100 Å; mobile phase: acetonitrile/water (with 0.25 mL/L trifluoroacetic acid) 63/37 (v/v), isocratic elution; oven temperature 40 °C; flow rate: 2.0 mL/min; run time 6 min, injection volume 1 μl.

### b) for Quantification of (V)

Column: 100×4.6 mm, stainless steel; stationary phase: Daicel, Chiracel OZ-3; mobile phase: heptane/ethanol 90/10 (v/v), isocratic elution; oven temperature 40 °C; flow rate: 1.0 mL/min; run time 10 min, injection volume 5 μl. An instrument with UV detection and external standard quantification was used.

### Example I: Preparation of 3-(3,5-Difluorophenyl)-5-Vinyl-4H-Isoxazole-5-Carboxylic Acid According to the Invention

### Step 0-1: Isopropyl 3-(3,5-difluorophenyl)-5-(1-hydroxyethyl)-4H-isoxazole-5-carboxylate

A suspension of 500 g of 3-(3,5-difluorophenyl)-5-(1-hydroxyethyl)-4H-isoxazole-5-carboxylic acid (1808 mmol, purity 98.1% by weight) in 1100 g of 2-propanol (99.0%) at 20 °C was heated to internal temperature 50 °C 260.1 g of thionyl chloride (2176 mmol, 99.5%) was added with a metering pump within 3 h. Subsequently, the solution was left to react further at 50 °C for a further 3 h. At the end of the reaction, a solid precipitated out of the solution, especially after the suspension had been cooled to room temperature. The conversion of 3-(3,5-difluorophenyl)-5-(1-hydroxyethyl)-4H-isoxazole-5-carboxylic acid or the formation of isopropyl 3-(3,5-difluorophenyl)-5-(1-hydroxyethyl)-4H-isoxazole-5-carboxylate can be analysed by HPLC. The yield of the desired isopropyl 3-(3,5-difluorophenyl)-5-(1-hydroxyethyl)-4H-isoxazole-5-carboxylate was >98%.

### Step 1

### Example 1.a

In a 250 ml four-necked flask equipped with reflux condenser, 50 g of isopropyl 3-(3,5-difluorophenyl)-5-(1-hydroxyethyl)-4H-isoxazole-5-carboxylate as a suspension in 66 ml of acetonitrile was initially charged at 20 °C. The mixture was purged with nitrogen. Subsequently, a total of 12.86 g of BF₃ gas (4.5 L; 1.2 eq) was introduced over the liquid surface. The pressure was equalized via an air balloon. The gas dissolved rapidly in the mixture; after adding ca. 4 g, a solution formed. The gas was added over 30 minutes. The solution was then stirred for 35 minutes and 60.92 g of N,N-dimethylcyclohexylamine (3 eq) was added slowly. The temperature was held at 20 °C. After 1 h, 17.75 g of SO₂F₂ (4.1 L; 1.09 eq) were added and the mixture heated to 80 °C. In total, the mixture was then kept at 80 °C for 19 h.

The sample was analyzed by HPLC; 98% of isopropyl 3-(3,5-difluorophenyl)-5-vinyl-4H-isoxazole-5-carboxylate was found.

### Example 1.b

In a 250 mL reactor equipped with a reflux condenser, 124.9 g of a suspension of isopropyl 3-(3,5-difluorophenyl)-5-(1-hydroxyethyl)-4H-isoxazole-5-carboxylate in xylene (38.4% by weight) were initially charged under nitrogen and 10 g of acetonitrile were added. Then, at 20 °C, 82.5 g of BF₃ acetonitrile solution (15.2% in acetonitrile) were added dropwise over 20 min. After addition was complete, the resulting solution was further stirred at 20 °C for 10 minutes and subsequently 78.0 g of N,N-dimethylcyclohexylamine (4 eq) were added dropwise over 2 h so that a temperature of 25 °C was not exceeded. Lastly, 20.0 g of SO₂F₂ (1.2 eq) were metered in below the liquid level at 20-25 °C over 4 h, after complete addition of which the reaction mixture was further stirred at 20 °C for 10 min and then heated to 80 °C over 6 h and further stirred at this temperature for 15 h.

A sample was analyzed by HPLC; >99% of isopropyl 3-(3,5-difluorophenyl)-5-vinyl-4H-isoxazole-5-carboxylate was found.

### Step 2

### Example 2.a

The mixture from example 1.a was distilled at an internal temperature of 75 °C and up to 90 mbar. Hydrolysis was effected with 126.4 g of NaOH (20% by weight, 4 equivalents). The solution was stirred at 65 °C for 25 minutes until hydrolysis was complete. HLPC analysis after acidification of a sample of the reaction mixture gave 98% 3-(3,5-difluorophenyl)-5-vinyl-4H-isoxazole-5-carboxylic acid.

### Example 2.b

86 g of acetonitrile were removed by distillation from the mixture of example 1.b at 80 °C up to 300 mbar and the reaction mixture was diluted with 80 g of xylene. After addition of 156 g of water, the phases were separated and the aqueous phase was extracted again with 25 g of xylene. The combined organic phases were treated with 156 g of water, 9.2 g of isopropanol and 61.4 g of NaOH (20% by weight, 2.0 eq). The reaction mixture was stirred at 50 °C for 8 h such that the completion of hydrolysis was established by HLPC analysis and, after acidification of a sample of the reaction mixture, gave 98% 3-(3,5-difluorophenyl)-5-vinyl-4H-isoxazole-5-carboxylic acid. With regard to the disposal thereof, the aqueous phase separated off may be further processed. For this purpose, potassium hydroxide or another potassium base may be added thereto up to a pH>9. The resulting potassium tetrafluoroborate salt (KBF₄) is separated off by filtration; fluoride may additionally be removed as required with agents commonly used by those skilled in the art.

### Workup

### Example 3.a

On completion of hydrolysis of the mixture of example 2.a, the mixture was distilled at 53-56 °C under reduced pressure, in order to remove N,N-dimethylcyclohexylamine by azeotropic distillation, and the lower water phase from the distillate was repeatedly fed to the reaction mixture.

The reaction mixture after distillation was added to 2.5 eq of hydrochloric acid in 100 mL of water, cooled in an ice bath. The product initially separated out as an oil and crystallized overnight at RT to give a solid. The solid was filtered off under suction and washed with 250 mL of water and then air-dried. 40.95 g of the desired 3-(3,5-difluorophenyl)-5-vinyl-4H-isoxazole-5-carboxylic were isolated. The purity was determined by quantitative HPLC and was 95% (corresponds to a yield of 97%).

### Example 3.b

After completion of hydrolysis and cooling of the mixture of example 2.b to 20 °C, the phases were firstly separated and the organic phase discarded. The aqueous phase was then adjusted to pH 1-2 by adding 32% by weight hydrochloric acid and the product extracted three times with a total of 130 g of tert-butyl methyl ether. The combined organic extracts were fully concentrated at 40 °C under reduced pressure and the product was isolated as a solid. 38.6 g of the desired 3-(3,5-difluorophenyl)-5-vinyl-4H-isoxazole-5-carboxylic were isolated. The purity was determined by quantitative HPLC and was 96.1% (corresponds to a yield of 96%).

The NMR data of the isolated and purified products and intermediates were determined as follows:

### Isopropyl 3-(3,5-difluorophenyl)-5-(1-hydroxyethyl)-4H-isoxazole-5-carboxylate (after step 0-1)

¹H-NMR (400 MHz, CDCl₃): δ (ppm)=1.28-1.32 (m, 9H), 2.18 (s, 1H), 3.53 (d, J=17.4 Hz, 1H), 3.67 (d, J=17.4 Hz, 1H), 4.22 (q, J=6.5 Hz, 1H), 5.13 (hept, J=6.3 Hz, 1H), 6.84-6.91 (m, 1H), 7.15-7.22 (m, 2H).

¹⁹F-NMR (376 MHz, CDCl₃): δ (ppm)=−108.4 (m, 2F).

### Isopropyl 3-(3,5-difluorophenyl)-5-vinyl-4H-isoxazole-5-carboxylate (after step 1)

¹H-NMR (401 MHz, CDCl₃): δ (ppm)=1.31 (dd, J=6.3, 1.0 Hz, 6H), 3.31 (d, J=17.0 Hz, 1H), 3.89 (d, J=17.0 Hz, 1H), 5.11 (hept, J=6.3 Hz, 1H), 5.36 (d, J=10.7 Hz, 1H), 5.54 (d, J=17.2 Hz, 1H), 6.13 (dd, J=17.2, 10.7 Hz, 1H), 6.84-6.90 (m, 1H), 7.15-7.22 (m, 2H).

¹⁹F-NMR (376 MHz, CDCl₃): δ (ppm)=−108.4 (m, 2F).

### 3-(3,5-Difluorophenyl)-5-vinyl-4H-isoxazole-5-carboxylic acid (after step 2)

¹H-NMR (400 MHz, CDCl₃): δ (ppm)=3.40 (d, J=17.1 Hz, 1H), 3.92 (d, J=17.1 Hz, 1H), 5.44 (d, J=10.7 Hz, 1H), 5.63 (d, J=17.2 Hz, 1H), 6.16 (dd, J=17.2, 10.7 Hz, 1H), 6.86-6.92 (m, 1H), 7.14-7.21 (m, 2H), 9.61 (bs, 1H).

¹⁹F-NMR (376 MHz, CDCl₃): δ (ppm)=−108.0 (m, 2F).

### Example II: Preparation of 3-(3,5-difluorophenyl)-5-vinyl-4H-isoxazole-5-carboxylic acid With and without Lewis Acid

To carry out a comparison, the method described in example 1.a, 2.a and 3.a was used but without Lewis acid in reaction mixtures 2 and 3. Acetonitrile was used as solvent.

The respective yields of the desired isopropyl 3-(3,5-difluorophenyl)-5-vinyl-4H-isoxazole-5-carboxylate (I) and, after hydrolysis, of the desired 3-(3,5-difluorophenyl)-5-vinyl-4H-isoxazole-5-carboxylic acid (V) are given in the table below in the column Yield I, %. Yields were measured in examples 4 to 6, in which the reaction temperature was successively raised during the reaction (room temperature (RT), 80 °C, 100 °C, in each case held for the times stated in the “time” column), at key points during the reaction course according to the reaction times and temperatures stated in the respective rows.

| | Substrate (IV) | | | | | | Conversion | | | |
|---|---|---|---|---|---|---|---|---|---|---|
| | X², X⁴, X⁶ = H; | | | | | | of IV, | II, | Yield I | VI, |
| | X³, X⁵ = F; | SO₂F₂, | Base, | Lewis | T, | Time, | HPLC | HPLC | HPLC | HPLC |
| Ex. | R² = H | eq | eq | acid, eq | °C | h | area % | area % | area % | area % |
| 1 | R¹ = isoPr | 1.09 | Me₂NCy, 3 | BF₃, | 80 | 19 | 100 | 0 | 98 | 0 |
| | | | | 1.2 | | | | | (97*) | |
| 2 | R¹ = isoPr | 1.3 | Me₂NCy, 2 | — | RT | >24 | 100 | 0 | 70 | 30 |
| 3 | R¹ = Me | 2 | Me₂NCy, 4 | — | RT | 15 | 100 | 0 | 61** | 39** |
| 4 | R¹ = isoPr | 1.3 | Me₂NCy, 4 | BF₃•Et₂O, | RT | 16 | 87 | 0 | 87 | <1 |
| | | | | 1.25 | 80 | 12 | 100 | | >99 | |
| 5*** | R¹ = isoPr | 1.3 | Me₂NCy, 4 | BF₃•Et₂O, | RT | 25 | n.d. | <1 | n.d. | <1 |
| | | | | 1.25 | 80 | 2.5 | 97 | | 96 | |
| | | | | | 100 | | 99 | | 98.5 | |
| 6 | R¹ = isoPr | 1.2 | Me₂NCy, 3 | BF₃ in | RT | 16 | 71 | <1 | 71 | <1 |
| | | | | CH₃CN, | 80 | 16 | >99 | | 99 | |
| | | | | 1.2 | | | | | | |

*The number in brackets is the isolated yield of the acid (V).
**Yield of (V) instead of (I), HPLC area %, after hydrolysis according to step 2 described above by way of example
***The reaction was carried out under 3.5 bar in a glass autoclave.

Whereas the presence of the Lewis acid in reaction mixture 1, and 4 to 6 according to the present invention results in a high HPLC yield and up to 97% isolated yield of the desired product (V), 3-(3,5-difluorophenyl)-5-vinyl-4H-isoxazole-5-carboxylic acid, the yields without Lewis acid (reaction mixtures 2 and 3) in the HPLC are at maximum 70% with high amounts of undesirable by-product VI.

1. A method for preparing compounds of formula (I) 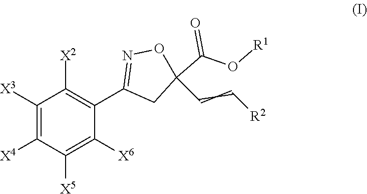
in which
R¹ is C₁-C₈-alkyl, cycloalkyl, unsubstituted benzyl, unsubstituted phenyl or mono- or di-C₁-C₃-alkyl-substituted benzyl or phenyl,
R² is H or alkyl,
X² is H, C₁-C₄-alkyl, C₁-C₄-fluoroalkyl, C₁-C₄-fluoroalkoxy, C₁-C₄-alkoxy, fluorine or CN,
X³ is H, C₁-C₄ alkyl, C₁-C₄ fluoroalkyl, C₁-C₄ fluoroalkoxy, C₁-C₄ alkoxy, fluorine, chlorine or CN,
X⁴ is H, C₁-C₄-alkyl, C₁-C₄-fluoroalkyl, C₁-C₄-fluoroalkoxy, C₁-C₄-alkoxy, fluorine or CN,
X⁵ is H, C₁-C₄ alkyl, C₁-C₄ fluoroalkyl, C₁-C₄ fluoroalkoxy, C₁-C₄ alkoxy, fluorine, chlorine or CN,
X⁶ is H, C₁-C₄-alkyl, C₁-C₄-fluoroalkyl, C₁-C₄-fluoroalkoxy, C₁-C₄-alkoxy, fluorine or CN,
the method comprising:
reacting, in a chemical reaction, compounds of general formula (IV) 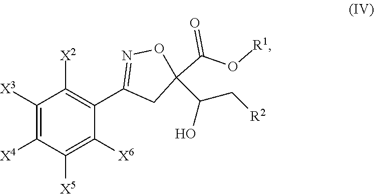
in which R¹, R², X², X³, X⁴, X⁵ and X⁶ have definitions given above,
with a compound of formula R^{F}SO₂F in the presence of a base and a Lewis acid,
where R^{F} is selected from the group consisting of F, CF₃, C₂F₅, C₃F₇, C₄F₉, CF₂Cl, C₆F₅, alkoxy-CF₂, and R⁶OCOCF₂SO₂F with R⁶ defined as C₁-C₈-alkyl or cycloalkyl.

2. The method according to claim 1, characterized in that the base is selected from the group consisting of trimethylamine, triethylamine, tripropylamine, tributylamine, N,N-diisopropylethylamine, N,N-dimethylcyclohexylamine, 2-methyl-5-ethylpyridine, pyridine, 3,5-dimethylpyridine, 2,4,6-trimethylpyridine, 2-methylpyridine, 3-methylpyridine, N,N-dimethylacetamide, N,N-dimethylformamide, N,N-dibutylformamide, N-methylimidazole (NMI), N-butylimidazole (NBI), 1,3-dimethyl-2-imidazolidinone (DMEU), and tetramethylurea (TMU).

3. The method according to claim 2, characterized in that the base is selected from the group consisting of triethylamine, tripropylamine, tributylamine, N,N-diisopropylethylamine and N,N-dimethylcyclohexylamine.

4. The method according to claim 1, characterized in that the base is used in amounts between 1.8 and 10.0 equivalents, based on one equivalent of the compounds of the formula (IV).

5. The method according to claim 1, characterized in that the Lewis acid is selected from the group consisting of BF₃, PF₅, and SbF₅.

6. The method according to claim 5, characterized in that the Lewis acid is BF₃.

7. The method according to claim 1, characterized in that the Lewis acid is used in amounts between 0.8 and 3.0 equivalents, based on one equivalent of the compounds of the formula (IV).

8. The method according to claim 1, characterized in that R^{F} is F.

9. The method according to claim 1, characterized in that R^{F}SO₂F is used in amounts between 0.8 and 4.0 equivalents, based on one equivalent of the compounds of the formula (IV).

10. The method according to claim 1, characterized in that the method is carried out in a solvent selected from the group consisting of acetonitrile, propionitrile, butyronitrile, acetone, N,N-dimethylacetamide, N-methylpyrrolidinone (NMP), N,N-dimethylformamide (DMF), dimethylpropyleneurea (DMPU), toluene, xylene, tetrahydrofuran (THF), isopropyl acetate (iPrOAc), dimethyl carbonate, diethyl carbonate, ethylene carbonate, propylene carbonate (4-methyl-1,3-dioxolan-2-one), ethyl acetate (EtOAc), methyl tert-butyl ether (MTBE), cyclopropyl methyl ether (CPME), methyl-THF, 4-methyltetrahydropyran (methyl-THP), tert-amyl methyl ether (TAME), dihydrolevoglucosenone (cyrene), N,N-dimethylcyclohexylamine and mixtures thereof in any ratios.

11. The method according to claim 1, characterized in that the method is carried out at least partially at a temperature between 60 °C to 100 °C.

12. The method according to claim 1, further comprising preparation of compounds of formula (IV) by reaction of compounds of formula (III) 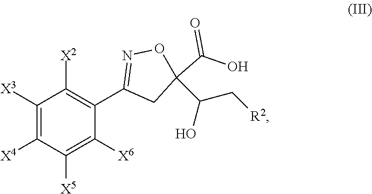
with compounds of formula R¹—OH,
in which R¹, R² and X² to X⁶ have the definitions given in claim 1.

13. The method according to claim 1, further comprising a reaction of the compounds of the formula (I) 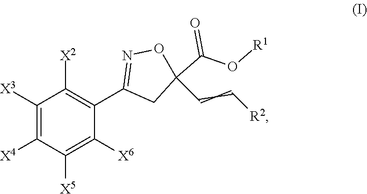
to give compounds of formula (V) 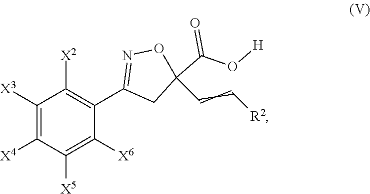
in which R¹, R² and X² to X⁶ have the definitions given in claim 1, in the presence of a base or acid.

14. The method according to claim 1, characterized in that
X² is H, methyl, trifluoromethyl, difluoromethyl, difluoromethoxy, trifluoromethoxy, fluorine, methoxy or CN;
X³ is H, methyl, trifluoromethyl, difluoromethyl, difluoromethoxy, trifluoromethoxy, fluorine, chlorine, methoxy or CN,
X⁴ is H, methyl, trifluoromethyl, difluoromethyl, difluoromethoxy, trifluoromethoxy, fluorine, methoxy or CN,
X⁵ is H, methyl, trifluoromethyl, difluoromethyl, difluoromethoxy, trifluoromethoxy, fluorine, chlorine, methoxy or CN,
X⁶ is H, methyl, trifluoromethyl, difluoromethyl, difluoromethoxy, trifluoromethoxy, fluorine, methoxy or CN.

15. The method according to claim 14, characterized in that
X² is H,
X³ is H or fluorine,
X⁴ is H or fluorine,
X⁵ is H or fluorine, and
X⁶ is H.

16. The method according to claim 4, characterized in that the base is used in amounts between 2.0 and 5.0 equivalents, based on one equivalent of the compounds of the formula (IV).

17. The method according to claim 6, characterized in that the BF₃ is introduced as a gas or a solution in acetonitrile.

18. The method according to claim 7, characterized in that the Lewis acid is used in amounts between 1.0 and 1.5 equivalents, based on one equivalent of the compounds of the formula (IV).

19. The method according to claim 9, characterized in that R^{F}SO₂F is used in amounts between 1.0 and 3.0 equivalents, based on one equivalent of the compounds of the formula (IV).
